(19) United States
(12) Patent Application Publication
(10) Pub. No.: US 2006/0275890 A1
(43) Pub. Date: Dec. 7, 2006

(54) Method of manufacturing a disposable diagnostic meter

(75) Inventors: Gary T. Neel, Weston, FL (US); Brent E. Modzelewski, Boca Raton, FL (US); Cameron Scott Casterline, Pembroke Pines, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Home Diagnostics, Inc.

(21) Appl. No.: 11/144,715

(22) Filed: Jun. 6, 2005

(51) Int. Cl. C12M 1/34; A61M 31/00

(52) U.S. Cl. 435/287.2; 427/2.11; 604/500

(57) ABSTRACT

A method for manufacturing a diagnostic testing system is provided. Various methods are described for calibrating the test system to work with selected test media compatible with the calibration to provide accurate results. The methods eliminate the need for any kind of user-coding. Packaging only compatible selected diagnostic test media with the calibrated meter can include at least one container for enclosing the diagnostic test media, wherein the container can be physically coupled to the diagnostic meter.

### TECHNICAL FIELD

The present invention relates to the field of diagnostic testing and, more particularly, to diagnostic testing systems using electronic meters.

### BACKGROUND

Electronic testing systems are commonly used to measure or identify one or more analytes in a sample. Such testing systems can be used to evaluate human body fluids for diagnostic purposes and to test various non-medical samples. For example, medical diagnostic meters can provide information regarding the presence, amount, or concentration of various analytes in human or animal body fluids. In addition, non-medical diagnostic test meters can be used to monitor analytes or chemical parameters in water, soil, sewage, sand, air, or any other suitable sample. Both medical and non-medical devices can also be configured to measure control solution for quality control.

Some diagnostic testing systems can be provided as integrated test kits, which can include a test media, a meter, and/or a sampling device. The test media (e.g., a test strip, tab, disc, drum, cylinder, etc.) can be configured to react to the presence of one or more analytes in a sample, and an electronic meter can be configured to interface with the test media in order to conduct the diagnostic test. Furthermore, a sampling device can be provided to obtain a sample from an appropriate source, such as capillary blood. Such integrated diagnostic test kits can conveniently provide all the needed components in one packaged kit. However, the currently available diagnostic test kits do present some problems.

Some diagnostic testing kits are bulky and cumbersome. Further, because the user must pick up and put down the test media container, sampling device and meter in succession, the test media container, sampling device and meter are often separated from each other. Consequently, users may find themselves without one or more of the components necessary to conduct the diagnostic test. Thus, it may be inconvenient for the user to carry a separate test media container, electronic meter and sampling device.

Further, test media from different brands or manufacturing lots may respond differently to the presence or concentration of analytes in a sample. In order to obtain more accurate results, the electronic meter may be calibrated with respect to a given brand or lot of test media. The meter may be calibrated by providing it with one or more brand or lot-specific calibration parameters that correlate the response from a particular brand or lot of test media to a standardized reference.

The user may be required to provide the meter with the appropriate calibration parameters in a separate “coding” step. For example, the test media container may display a code number from which the meter can determine the appropriate calibration information. The user can manually enter the code number (e.g., using buttons or other user input devices on the meter) so as to provide the calibration data to the meter. Alternatively, the calibration data may be downloaded, e.g., from a manufacturer's website. In another approach, the test media container can be provided with an associated calibration data chip, which the user can insert into a port on the meter to load the calibration data.

This coding step can be inconvenient or difficult for the user. For example, elderly or infirm users may have difficulty entering or downloading calibration data or inserting code chips. Further, users may forget to calibrate the meter for use with a new brand or lot of test media. Consequently, the user may enter incorrect calibration parameters or codes, or the user may use test media from one brand or lot with a meter calibrated for use with test media from a different brand or lot. However, once a meter is calibrated for a given lot of test media, the use of that meter with test media from another lot may lead to erroneous results that could have serious consequences for the user. For example, where the test is a self-test of blood glucose concentration, an erroneous result can misinform the user as to their blood glucose level, which can lead to serious health problems from hypo- or hyperglycemia.

Accordingly, there is a need for diagnostic testing systems that are convenient to carry and that minimize the chance that a user will use a diagnostic meter with test media from a brand or lot for which the meter has not been calibrated.

### SUMMARY

One aspect of the present disclosure includes a method for manufacturing a diagnostic testing system. The method can include producing a diagnostic meter configured to measure the concentration of one or more analytes in a sample, selecting one or more diagnostic test media for use with the diagnostic meter, calibrating the diagnostic meter to configure the meter to measure the concentration of one or more analytes using the selected diagnostic test media, and packaging the selected diagnostic test media with the meter and at least one container for enclosing the diagnostic test media, wherein the container can be physically coupled to the diagnostic meter.

A second aspect of the present disclosure includes a method for manufacturing a diagnostic testing system. The method can include producing a diagnostic meter configured to measure the concentration of one or more analytes in a sample, calibrating the diagnostic meter using predetermined calibration data, producing one or more test media, and selecting from the one or more test media at least one test strip configured to measure the concentration of one or more analytes using the diagnostic meter that has been calibrated with the predetermined calibration data.

A third aspect of the present disclosure includes a method for manufacturing a diagnostic testing system. The method can include producing a diagnostic meter configured to measure the concentration of one or more analytes in a sample, calibrating the diagnostic meter with a predetermined calibration data, and producing one or more test media specifically configured to measure the concentration of one or more analytes using the diagnostic meter that has been calibrated with the predetermined calibration data.

A fourth aspect of the present disclosure includes a diagnostic testing system. The system can include an enclosure, a set of diagnostic test media which can be disposed within the enclosure, and a diagnostic meter that can be physically coupled with the enclosure, wherein the diagnostic meter is configured to measure the concentration of one or more analytes in a sample and is calibrated to measure the concentration of the one or more analytes specifically using at least one of diagnostic test strip included in the set of diagnostic test media.

Additional aspects and advantages of the invention will be set forth in part in the description which follows, and in part will be apparent from the description, or can be learned by practice of the invention. The advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a first embodiment of an integrated system consistent with the present invention.

FIG. 2 is a perspective view of a second embodiment of an integrated system consistent with the present invention.

FIG. 3 is a perspective view of a third embodiment of an integrated system consistent with the present invention.

FIG. 4 provides a diagram of a method for manufacturing a diagnostic test system, according to an exemplary embodiment.

FIG. 5 provides a diagram of a method for manufacturing a diagnostic test system, according to an another exemplary embodiment.

FIG. 6 provides a diagram of a method for manufacturing a diagnostic test system, according to yet another exemplary embodiment.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

1. The Integrated System

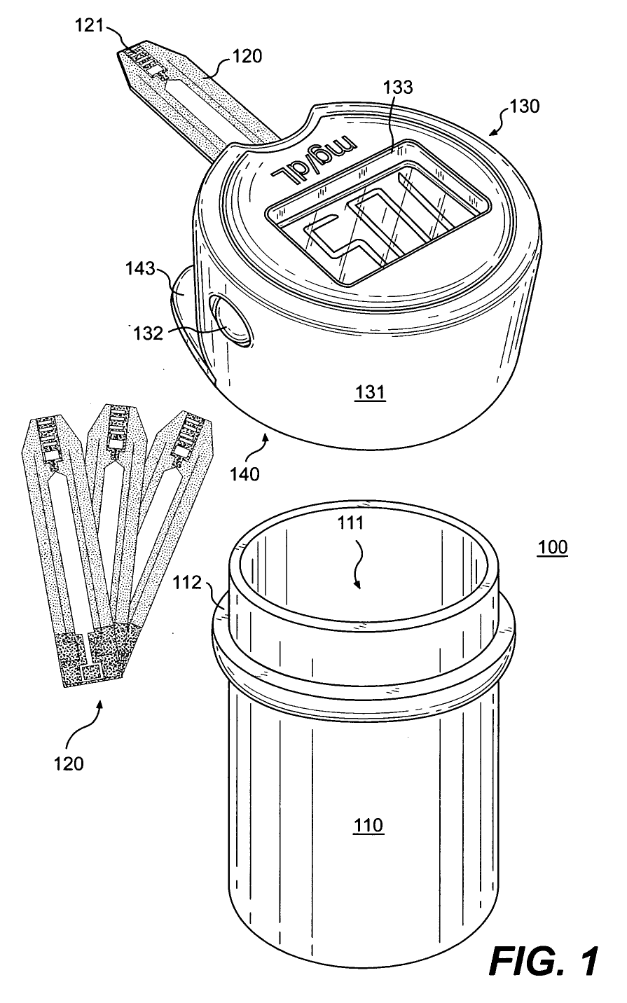

FIG. 1 shows an integrated system 100 for conducting a diagnostic test in accordance with an exemplary embodiment of the present invention. Exemplary integrated system 100 includes a container 110 for containing test media, such as test strips 120, and a meter 130 for performing a diagnostic test using test strips 120 contained in container 110.

Integrated system 100 may be used to provide diagnostic tests on a variety of suitable samples. For example, in some embodiments, the diagnostic test may include a test of a medical sample, including a body fluid from humans or animals. Such body fluids may include, for example, blood, serum, plasma, interstitial fluid, urine, cerebral-spinal fluid, saliva, sweat, tear, mucous, sputum, gastric fluid, feces, and any other suitable fluid. In addition, the diagnostic test may also include suitable non-medical samples including, for example, water, sewage, pool water, well water, soil, wine, beer, maple syrup, other food products, or any other suitable sample.

In one illustrative embodiment, the diagnostic test is the determination of the amount of glucose in a sample applied to a sample chamber 121 of test strip 120. In one embodiment, the sample can include blood. For blood glucose testing, meter 130 can employ any of a variety of techniques. Preferably, the diagnostic test employs an electrochemical technique (e.g., coulometry, amperometry, potentiometry, etc.). Exemplary electrochemical systems are described in prior application Ser. Nos. 10/286,648, filed Nov. 1, 2002, and 10/420,995, filed Apr. 21, 2003, both entitled “SYSTEM AND METHOD FOR BLOOD GLUCOSE TESTING” and both having assignee in common with the instant application, which are incorporated by reference herein in their entirety. Alternatively, meter 130 can employ a photometric technique (e.g., reflection, transmission, scattering, absorption, fluorescence, electro-chemiluminescence, etc.) to determine the amount of glucose in the sample. Exemplary photometric systems are described in U.S. Pat. Nos. 6,201,607, 6,284,550 and 6,541,266, each having assignee in common with the instant application, which are incorporated by reference herein in their entirety. However, electrochemical techniques are currently preferred because, among other things, they can require a smaller blood sample (on the order of 1 μL or less) than photometric techniques (on the order of 1 μL or greater). Further, the instrumentation for the electrochemical techniques typically requires less power and can be made more compact than the instrumentation for the photometric techniques.

Integrated system 100 will be illustrated with reference to a diagnostic test to determine the concentration of blood glucose using an electrochemical technique, with the understanding that the principles of the present invention are equally applicable to other types of diagnostic tests and techniques, such as those mentioned above. Further, although the present invention has been illustrated as utilizing test media in the form of test strips 120, exemplary embodiments of the present invention are not limited to a particular type of media, and those of skill in the art will recognize that the principles of the present invention are equally applicable to diagnostic testing systems which employ test media in other forms, e.g., tabs, discs, drums, cylinders, etc.

Meter 130 can be contained within a housing 131. Meter housing 131 can be attached to or otherwise include a closure portion 140 (bottom of meter 130 in FIG. 1) which engages container 110 in order to selectively close an opening 111 of container 110. Opening 111 can be the only opening in container 110. In an illustrative embodiment, meter housing 131 has one side (e.g., the bottom of meter housing 131 in FIG. 1) which is shaped to conform to closure portion 140 and is affixed to closure portion 140, e.g., by a mechanical attachment (clips, snaps, etc.), bonding, gluing, welding, etc. Alternatively, closure portion 140 can be formed integrally with meter housing 131. Meter 130 and closure portion 140 together thus form a cap or lid for the container 110.

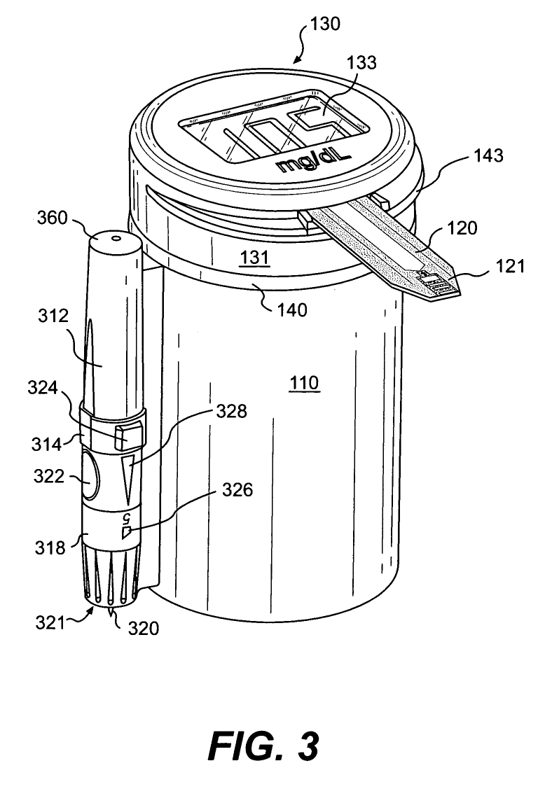

Closure portion 140 can be configured to engage container 110 in a number of ways. In the closed position (see FIG. 3), closure portion 140 closes opening 111 sufficiently to prevent loss or removal of the test media from container 110. Accordingly, closure portion 140 can be configured to engage container 110 so as to prevent test strips 120 from passing through opening 111 when closure 140 is in the closed position. Container 110 and closure 140 can also be configured to prevent the infiltration of light, liquid, vapor, and/or air into container 110 so as to prevent contamination or degradation of the test media. Where the test media can be toxic or can present a choking hazard, closure 140 can optionally be configured to be child-resistant in order to prevent children from opening container 110 and accessing the test media. For example, closure 140 and container 110 can be configured in a manner similar to well known child-resistant containers for pharmaceuticals or household chemicals.

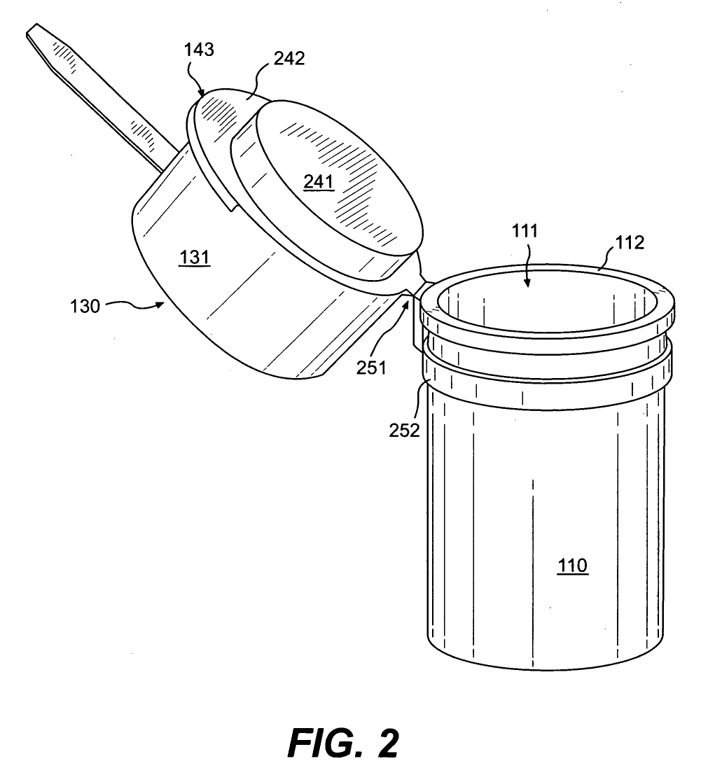

Closure 140 can be configured as a twist-off cap, e.g., by providing inter-engaging threads (not shown) on closure portion 140 and container 110. Alternatively, closure portion 140 can be configured to slide over the opening, e.g., within grooves (not shown) beside the opening. As a further alternative, closure portion 140 can be provided with a catch (not shown), such as a detent, that engages container 110 (or vice versa). The catch can be released by a button. However, in one illustrative embodiment, closure portion 140 is configured to form a press-fit or interference-fit seal with container 110 so as to seal the opening to light, liquid and/or vapor. For example, in FIG. 1, closure portion 140 is configured with a recess (not shown) to press-fit to the outside of opening 111, so that the rim of opening 111 fits within closure portion 140. Alternatively, closure portion 140 can be configured with a projection 241 shaped to engage the inside of opening 111, as shown in FIG. 2. However, it will be understood that the present invention is not limited to any particular configuration of the container and closure, and other configurations can be employed consistent with the principles of the present invention.

For ease of manufacture, opening 111 can be made in the same shape as container 110. Housing 131 of meter 130 can likewise have an exterior shape similar to that of container 110 so that integrated system 100 can be more comfortably held and carried, e.g., in a user's pocket. However, it will be understood that container 110, meter 130 and opening 111 need not be of the same exterior shape, and container 110 and meter 130 can have different shapes without departing from the scope of the present invention.

Preferably, container 110 is generally a right circular cylinder and opening 111 has a circular shape as shown in FIGS. 1 and 2. A circular shape is one possible configuration for the opening because it allows a uniformly tight seal to be formed with a press-fit between closure portion 140 and container 110. As shown in FIGS. 1-3, meter 130 can also be generally circular and cylindrical and have a width similar to the width of container 110 so that integrated system 100 has an overall generally circular-cylindrical shape that is comfortable to hold and to carry, e.g., in a pants pocket. However, container 110, meter 130 and opening 111 can be made in any of a number of other shapes. For example, in order to better conform to a user's shirt pocket, container 110 can be formed as a right oval, elliptical or rectangular cylinder.

In order to further prevent the infiltration of liquid and/or gases, container 110 and closure portion 140 can also be provided with corresponding flanges 112 and 242, respectively, that can fit flush against each other when closure portion 140 is in the closed position. In addition, to aid the user in opening and closing container 110, closure portion 140 can be provided with a protrusion or “bill” 143 which extends laterally beyond the side of container 110 to allow the user to exert an upward with the thumb or fingers against protrusion 143. Protrusion 143 can be an extension of flange 242, as shown in FIG. 2, or alternatively, protrusion 143 can be formed directly on meter housing 131, as shown in FIG. 3. The protrusion 143 can be located at any convenient location about the periphery of meter housing 131, and can extend partially or entirely around. In place of protrusion 143, a knurl or other gripping surface can be used.

As shown in FIG. 1, container 110 can be opened by completely removing meter 130 and closure portion 140 from container 110. Alternatively, meter 130 and/or closure 140 can be connected to container 110 in order to prevent meter 130 from becoming separated from container 110. Container 110 and meter 130 can be connected by, e.g., a hinge, lanyard or other flexible connector, such as a flexible plastic band or wire, etc. (not shown). In an illustrative embodiment, a hinge 251 connects container 110 and meter housing 131 and/or closure portion 140. Hinge 251 can be positioned such that projection 241 fits within opening 111 in the closed position. The connector (e.g., hinge 251) can have one end connected to container 110 and the other end connected to closure portion 140 and/or meter housing 131. For example, container 110 and closure portion 140 can be integrally connected by a hinge, e.g., as shown in U.S. Pat. No. 5,723,085, entitled “PROCESS AND APPARATUS FOR MAKING A LEAK PROOF CAP AND BODY ASSEMBLY,” which is incorporated by reference herein in its entirety. Alternatively, one end of the connector (e.g., hinge 251) can be connected to a ring 252 that is sized to fit over container 110, as shown in FIG. 2. Ring 252 can be configured to loosely frictionally engage container 110. As another alternative, ring 252 can be affixed to container 110, e.g., by welding, gluing, etc.

In an exemplary embodiment, container 110 and closure 140 are formed of polypropylene using an injection molding process. However, other materials and processes can be used without departing from the scope of the present invention.

Integrated system 100 can further include a sampling device which can be used to obtain a sample for testing. The sampling device can be adapted to obtain a biological sample. For instance, the sampling device can be a lancing device that the user can use to draw blood, e.g., for a diagnostic test of blood glucose level.

An exemplary integrated system 100 incorporating a lancing device 360 is shown in FIG. 3. Exemplary lancing device 360 includes a rearward body 312, a finger cover 314, an exterior nozzle 318, an interior nozzle 322 and a trigger 324. Exemplary lancing device 360 further includes an internal spring (not shown) that is used to propel a lancet 320 beyond a contact surface 321 and through the skin to depth selected by a user.

As shown in FIG. 3, exemplary lancing device 360 can be connected to container 110. Lancing device 360 can be permanently connected to container 110, for instance, by forming, e.g., rearward body 312, finger cover 314, exterior nozzle 318 or interior nozzle 322 integrally with the container 110, or by bonding one of these components to container 110, e.g., by a mechanical attachment (clips, etc.), bonding, gluing, welding, etc. Alternatively, lancing device 360 can be releasably connected to container 110 by providing corresponding releasable connectors on lancing device 360 and container 110. For example, lancing device 360 can be provided with one or more slots, holes or clips that engage corresponding structures on container 110, or vice versa. As further alternatives, lancing device 360 can be connected to housing 131 of meter 130, or to closure portion 140. Preferably only one of rearward body 312, finger cover 314, exterior nozzle 318 or interior nozzle 322 is connected to container 110 so that lancing device 360 can be adjusted and used without disconnecting it from container 110.

In order to draw a sample using exemplary lancing device 360, the user can first select a desired depth of penetration of lancet 320 by rotating exterior nozzle 318 so that a depth indicator 326 on exterior nozzle 318 is aligned with an arrow 328 on interior nozzle 322. Next, the user loads the internal spring by pulling interior nozzle 322 away from rearward body 312. The user then places contact surface 321 against the surface to be lanced and actuates trigger 324 to release the internal spring to propel lancet 320 beyond contact surface 321 to the indicated depth, and thus into the skin. A blood sample can then be applied to sample chamber 121 of test strip 120.

Further details of exemplary lancing device 360 are shown in prior application Ser. No. 10/757,776, entitled “LANCING DEVICE,” filed Jan. 15, 2004, having assignee in common with the instant application, which is incorporated by reference herein in its entirety. However, the present invention is not limited to any particular sampling device, and one of skill in the art will recognize that other sampling devices can be incorporated in a manner similar to the exemplary lancing device described above.

2. Method of Manufacturing and Calibrating

Meter 130 can be calibrated for use with a particular brand or manufacturer's lot of test media by customizing the diagnostic test performed by meter 130 with respect to the particular brand or lot using one or more calibration parameters. These calibration parameters can include environmental corrections (e.g., temperature, humidity, oxygen, altitude corrections), timing period configurations (e.g., with respect to the testing sequence), voltage corrections (e.g., for use in electrochemical tests), color variations (e.g., for use in radiometric tests), hematocrit levels in the blood, etc., that customize the diagnostic test function of controller 400 to the particular brand or lot of test media. See, e.g., application Ser. Nos. 10/286,648 and 10/420,995, incorporated by reference above.

Calibration of the meter 130 may have more than one meaning to those familiar with the art. For example, at the time of manufacturing meter 130, various meter components may be calibrated for electrical and device-specific parameters, such as current (in nano-Amps), dead battery voltage, low battery voltage, and other hardware/process parameters. This type of calibration can be termed “internal” calibration, meaning that the device hardware is characterized and conformed to an operational standard. However, the term calibration (also referred to as “coding”) may also refer to adjusting the meter 130 for use with specific test media (i.e. test media having a certain chemistry, kinetic properties, etc.). Calibration of meter 130 for use with specific test media (or test media lot) may include calibration with respect to a reference, such as a Yellow-Spring Instrument for glucose, or any other suitable reference, usually through the provision of calibration constants used by the device algorithm in making an analog conversion (e.g., current to analyte concentration). As described and used in the present disclosure, the terms “calibration” and “pre-calibration” should be understood to refer to calibration of the meter 130 for use with specific test media (or test media lots), such that meter 130 will provide an accurate diagnostic test when used with the specific test media. It should be understood that calibration of the device electronics and/or hardware would naturally have already been performed.

In an illustrative embodiment of the present invention, integrated system 100 includes one or more containers 110 of test strips 120 packaged together with meter 130. Test strips 120 in the package can be from the same manufacturing lot or otherwise have the same characteristic reaction to blood glucose so that meter 130 can be calibrated once and thereafter used with any of the test strips 120 in the package without recalibration. Furthermore, as described previously, the meter 130 can be permanently or removable coupled with one or more containers 110 of test strips 120.

Because the use of meter 130 with test media from a brand or lot for which the meter 130 has not been calibrated can lead to errors, exemplary embodiments of the present invention minimize the chance that a user will mistakenly use meter 130 with test media from a brand or lot for which meter 130 has not been calibrated. In an illustrative embodiment, the functional components of meter 130 are chosen and constructed such that meter 130 is economical to market as a disposable device. For example, meter 130 can be constructed using low-cost components, or one or more of the functional components of exemplary meter 130 described above can be omitted in order to reduce the overall cost of meter 130. Further, the test media and meter 130 can be packaged together such that the user receives a new meter 130 with each purchase of test media. Consequently, the user is encouraged to dispose of their old meter 130 when the test media packaged with meter 130 (e.g., in container 110) have been used up. In this manner, exemplary embodiments of the present invention reduce the likelihood that a user will mistakenly use meter 130 with test media from a brand or lot for which meter 130 has not been calibrated.

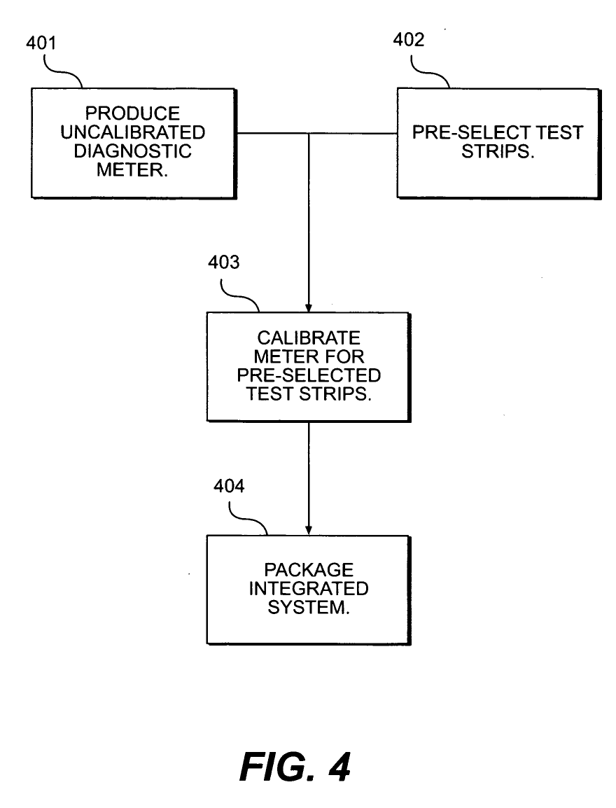

FIGS. 4-6 provide diagrams of methods for manufacturing integrated system 100, according to exemplary disclosed embodiments. In each of the embodiments of FIGS. 4-6, meter 130 can be calibrated during manufacturing, such that integrated system 100 will include one or more test strips 120 or other test media that can be used with meter 130 to measure or identify one or more analytes in a sample.

FIG. 4 illustrates one method for manufacturing integrated system 100, according to an exemplary disclosed embodiment. In this embodiment, meter 130 can be produced as shown in step 401 without being calibrated or, alternatively with an adjustable calibration. Next, a manufacturer can select one or more test media as shown in step 402, which have been produced by any suitable procedure, such that the selected test media will provide substantially similar test results when used with the same test meter 130. For example, in one embodiment, one or more test media can be selected from a single test media manufacturing lot, which can be expected to yield test media having substantially similar functional properties.

Meter 130 will then be calibrated as shown in step 403 such that the meter 130 will provide an accurate diagnostic test when used with at least one of the one or more specific selected test media. Particularly, meter 130 can be calibrated to provide accurate test results when used with any of the one or more selected test media. Calibration can be effected using a number of suitable techniques. For example, meter 130 can be calibrated by configuring the hardware contained within meter 130 to function using the appropriate media, as selected according to the present invention. For example, in one embodiment, meter 130 can be calibrated by making suitable adjustments in one or more electrical circuits contained within meter 130. Alternatively or additionally, the meter 130 can be permanently or reversibly sealed after calibration of the meter hardware, thereby preventing inadvertent or intentional modification of the calibration parameters.

In another embodiment, meter 130 can include software functions, which can access data stored in suitable media within meter 130. For example, meter 130 can include various rewritable or permanently writable data units, including optical, magnetic, or electrical storage media. Calibration data can be loaded into the storage media during manufacturing, and the meter hardware and/or software can access the calibration data during testing. Further, the calibration data can be permanently written to the storage media such that a user cannot adjust the calibration data either accidentally or intentionally.

Finally, integrated system 110 can be packaged as shown in step 404 to include meter 130, test strips 120, and container 110, in which test strips 120 can be enclosed. Further, multiple containers 110 of test media with substantially similar functional properties can be packaged with a single meter. Particularly, a number of containers and/or test media can be packaged together to provide a desired number of diagnostic tests with a single meter.

Further, meter 130 can include various hardware and/or software configurations, which can limit the functional life of meter 130. For example, meter 130 can be packaged with a certain number of test strips 120, and meter 130 can be configured to perform a number of tests approximately equal to the number of test strips packaged with the meter 130. Meter 130 can be further configured to stop functioning after performing a certain number of tests. In a preferred embodiment, the meter will be configured to disable itself after n or more tests are run, where n is the number of strips packaged with the meter. In exemplary embodiments, n+1 or n+2 is used. The additional test(s) will account for the possibility that one or more extra strips will be included with the test meter. For example, additional test strips may be included accidentally due to human error or production process limitations. Alternatively, additional test strips may be included intentionally, e.g., for training purposes, marketing promotions, etc. Of course, n+3 could be used, or n+y for any integer value of y. Alternatively, or additionally, meter 130 can be configured to stop functioning after a certain time period, such as a specific number of days or months. In this way, a user can be prevented from using test strips 120, which may not have been provided with meter 130 and/or may not be appropriately calibrated for use with the meter 130. Additionally, the user can be prevented from using test strips 120 which may have aged significantly and may no longer provide accurate test results.

Various methods can be employed to disable the meter at the appropriate time. For instance, a software-based lockout, or a hardware-based system such as a fusible link or a battery drain could be used.

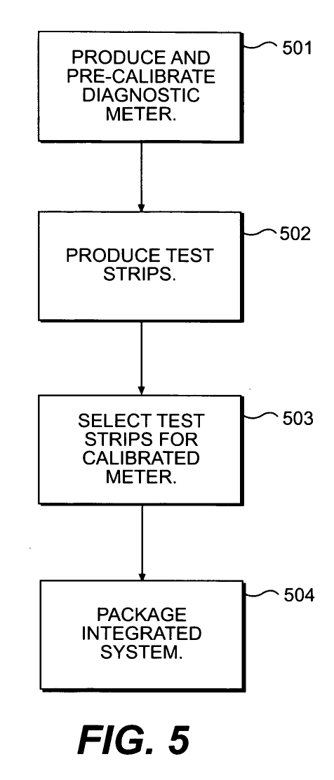

FIG. 5 illustrates another method for manufacturing integrated system 100, according to an exemplary disclosed embodiment. In this embodiment, meter 130 can be produced and then calibrated with predetermined calibration data as shown in step 501. The predetermined calibration data can be selected from a group of commonly useful calibration codes with respect to the test media to be used with the meter 130. Alternatively or additionally, all meters can be calibrated using the same calibration data.

Next, in step 502, a group of test strips or other suitable test media can be produced using any suitable production process. In some production processes, there may be an inherent variation in the functional properties among the test media produced. Therefore, in this embodiment, unlike in the embodiment of FIG. 4, it may be necessary to select one or more test media from among all the test media produced which will provide accurate diagnostic tests when used with meter 130 having compatible predetermined calibration data, as shown in step 503. This process is essentially the reverse of calibrating a meter for use with selected test media. After selecting the test strips, the strips and meter can be packaged, as shown at step 504.

The one or more test media having the desired properties compatible with the calibration data pre-installed in the meter can be selected in a number of suitable ways. For example, in one embodiment, one or more lots of test strips can be produced, and each lot can contain a number of test strips, which can be expected to have substantially similar functional properties. One or more test media from each lot can be evaluated to determine the functional properties of all the test media within the lot, and if the one or more test media in the lot are found to have the desired properties (i.e. being capable of providing accurate diagnostic tests when used with the meter 130 having compatible predetermined calibration parameters) then one or more additional test media from the same lot can be selected for use with the calibrated meter.

It should be noted that in the embodiment of FIG. 5, the predetermined calibration can be selected from a set of one or more suitable calibrations. In one embodiment, a certain number of test meters 130 can be produced, and a certain fraction of the meters can be calibrated with each of the one or more suitable calibrations. For example, using a particular test media manufacturing process, the inherent variation in the manufacturing process can be characterized, and a certain set of calibration codes can be identified to correspond with the test media produced. Further, the meters 130 can be calibrated using a proportion of the identified calibration codes, such that the number of test meters 130 having a certain calibration corresponds with a certain proportion of test media that can function properly using the calibrated meters 130. In this way, the manufacturer can produce test media with lot-to-lot variations in functional properties, but can still use most or all test media without having to discard substantial number of test media due to normal variations in manufacturing.

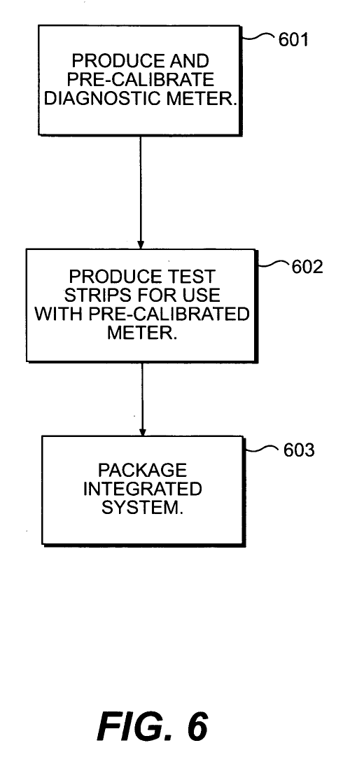

FIG. 6 illustrates another method for manufacturing integrated system 100, according to an exemplary disclosed embodiment. In this embodiment, multiple meters 130 can be produced and calibrated with the same predetermined calibration code as shown in step 601. Next, in step 602, a set of test media can be produced such that all the test media can provide accurate test results when used with any meter 130 having the single predetermined calibration. The strips and meter are packaged together in step 603.

The test media can be produced using a number of suitable processes. For example, each of the test media can be produced using the same process, and the process parameters, including for example, materials deposition, etching, ablation, temperature, pressure, etc. can be closely monitored and controlled to provide test media having desired functional properties. Particularly, the production process can be selected to provide test media that will provide accurate test results when used with any test meter 130 having a predetermined calibration.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for manufacturing a diagnostic testing system, comprising:
producing a diagnostic meter configured to measure the concentration of one or more analytes in a sample;
pre-selecting one or more diagnostic test media having substantially similar functional properties for use with the diagnostic meter;
calibrating the diagnostic meter such that, in use, said meter is capable of accurately measuring the concentration of one or more analytes using the pre-selected diagnostic test media; and
packaging the pre-selected diagnostic test media with the calibrated meter and at least one container for enclosing the diagnostic test media, wherein the container can be physically coupled to the diagnostic meter.

2. The method of claim 1, wherein the sample comprises body fluid.

3. The method of claim 2, wherein the sample is blood.

4. The method of claim 1, wherein the sample includes a non-medical sample.

5. The method of claim 4, wherein the sample includes water.

6. The method of claim 1, wherein the one or more analytes includes glucose.

7. The method of claim 1, wherein calibrating the diagnostic meter includes configuring the test meter software to allow the meter to accurately measure the concentration of one or more analytes using the pre-selected diagnostic test media.

8. The method of claim 1, wherein calibrating the diagnostic meter includes configuring the test meter hardware to allow the meter to accurately measure the concentration of one or more analytes using the pre-selected diagnostic test media.

9. The method of claim 1, further including configuring the diagnostic test meter to stop functioning after performing a number of diagnostic tests no less than the number of test media included in the one or more test media.

10. A method for manufacturing a diagnostic testing system, comprising:
producing a diagnostic meter configured to measure the concentration of one or more analytes in a sample;
pre-calibrating the diagnostic meter using one set of predetermined calibration data;
producing a plurality of test media; and
selecting from the plurality of test media at least one test strip only if compatible with said one set of predetermined calibration data and thereby configured to accurately measure the concentration of one or more analytes using the diagnostic meter that has been pre-calibrated with the predetermined calibration data.

11. The method of claim 10, wherein the sample comprises body fluid.

12. The method of claim 11, wherein the sample is blood.

13. The method of claim 10, wherein the one or more analytes includes glucose.

14. The method of claim 10, wherein the sample includes a non-medical sample.

15. The method of claim 14, wherein the sample includes water.

16. The method of claim 10, wherein pre-calibrating the diagnostic meter includes configuring the test meter software to allow the meter to accurately measure the concentration of one or more analytes using the selected diagnostic test media.

17. The method of claim 10, wherein calibrating the diagnostic meter includes configuring the test meter hardware to allow the meter to measure the concentration of one or more analytes using the selected diagnostic test media.

18. The method of claim 10, further including configuring the diagnostic test meter to stop functioning after performing a number of diagnostic tests no less than the number of test media included in the one or more test media.

19. A method for manufacturing a diagnostic testing system, comprising:
producing a diagnostic meter configured to measure the concentration of one or more analytes in a sample;
pre-calibrating the diagnostic meter with one set of predetermined calibration data for each said analyte; and
producing one or more test media wherein all said test media are configured to accurately measure the concentration of one or more analytes using the diagnostic meter that has been pre-calibrated with the one set of predetermined calibration data.

20. The method of claim 19, wherein the sample comprises body fluid.

21. The method of claim 20, wherein the sample is blood.

22. The method of claim 19, wherein the sample includes a non-medical sample.

23. The method of claim 22, wherein the sample includes water.

24. The method of claim 19, wherein the one or more analytes includes glucose.

25. The method of claim 19, wherein pre-calibrating the diagnostic meter includes configuring the test meter software to allow the meter to measure the concentration of one or more analytes using the selected diagnostic test media.

26. The method of claim 19, wherein pre-calibrating the diagnostic meter includes configuring the test meter hardware to allow the meter to measure the concentration of one or more analytes using the selected diagnostic test media.

27. The method of claim 19, further including configuring the diagnostic test meter to stop functioning after performing a number of diagnostic tests no less than the number of test media included in the one or more test media.

28. A diagnostic testing system, comprising:
an enclosure;
a set of diagnostic test media which can be disposed within the enclosure; and
a diagnostic meter that can be physically coupled with the enclosure, wherein the diagnostic meter is configured to accurately measure the concentration of one or more analytes in a sample and is calibrated to accurately measure the concentration of the one or more analytes using at least one of diagnostic test strip included in the set of diagnostic test media.

29. The system of claim 28, further comprising a sampling device operably connected to the container.

30. The system of claim 29, wherein the sampling device comprises a lancet.

31. The system of claim 30, wherein the lancet comprises a spring for propelling the lancet to puncture the skin and the lancet is connected to the container such that a user can load the spring and release the spring to draw a sample without disconnecting the lancet from the container.

32. The system of claim 31, wherein the lancet comprises a mechanism for adjusting the depth of penetration of the lancet and the lancet is connected to the container and configured such that a user can operate the mechanism to adjust the depth of penetration of the lancet without disconnecting the lancet from the container.
